(19) United States
(12) Patent Application Publication
(10) Pub. No.: US 2025/0114326 A1
(43) Pub. Date: Apr. 10, 2025

(54) POLAR MARINE-DERIVED MACROCYCLIC LACTAM COMPOUND, AND PREPARATION METHOD THEREFOR AND USE THEREOF

(71) Applicant: RENJI HOSPITAL, SCHOOL OF MEDICINE, SHANGHAI JIAO TONG UNIVERSITY, Shanghai (CN)

(72) Inventors: Houwen LIN, Shanghai (CN); Fan YANG, Shanghai (CN)

(73) Assignee: RENJI HOSPITAL, SCHOOL OF MEDICINE, SHANGHAI JIAO TONG UNIVERSITY, Shanghai (CN)

(21) Appl. No.: 18/714,075

(22) PCT Filed: Nov. 25, 2022
(86) PCT No.: PCT/CN2022/134337

§ 371 Date: May 28, 2024

(30) Foreign Application Priority Data
Dec. 1, 2021 (CN) 202111454811.5

(51) Int. Cl. A61K 31/395; A61P 29/00; C07D 498/08; C07D 498/18; C12P 17/18; C12R 1/465

(52) U.S. Cl. CPC A61K 31/395; A61P 29/00; C07D 498/08; C07D 498/18; C12P 17/188; C12R 2001/465

(57) ABSTRACT

Provided in the present invention are a polar marine-derived macrocyclic lactam compound, and a preparation method therefor and the use thereof. Provided in the present invention is a macrocyclic lactam compound as represented by formula 1 or a pharmaceutically acceptable salt thereof. The compound has a novel structure and good inflammation inhibitory activity, and provides a new candidate compound for developing an anti-inflammatory drug.

The present application claims the priority of China Patent Application 2021114548115 filed on Dec. 1, 2021. This Chinese patent application is incorporated herein by reference in its entirety.

### TECHNICAL FIELD

The present disclosure relates to a polar marine-derived macrocyclic lactam compound, and a preparation method therefor and the use thereof.

### BACKGROUND

Inflammation is the body's defense mechanism against infection, primarily manifested by redness and swelling, pain, etc. There are two main types of drugs in the clinical treatment of inflammation: glucocorticoid anti-inflammatory drugs with steroidal structures, and non-steroidal anti-inflammatory drugs.

Glucocorticoid anti-inflammatory drugs, represented by cortisone drugs, came out in the 1940s and have achieved remarkable efficacies in the treatment of arthritis and other diseases. However, long-term use can lead to drug dependence and some severe side effects, including compromised adrenocortical function, which limits their widespread use in clinical practice.

The concept of non-steroidal anti-inflammatory drugs (NSAIDs) dates back to the 1950s. These drugs do not contain steroidal structures. Subsequently, research and development of anti-inflammatory drugs became a hot topic, and a series of non-steroidal anti-inflammatory drugs were successively developed and launched on the market. These drugs can be primarily divided into pyrazolones, anthranilic acids, indoleacetic acids, arylalkanoic acids, etc.

### CONTENT OF THE PRESENT INVENTION

The technical problem to be solved by the present disclosure is that the existing anti-inflammatory drugs have a single structure. Therefore, the present disclosure provides a polar marine-derived macrocyclic lactam compound, and a preparation method therefor and the use thereof. The compound has a novel structure and good inflammation inhibitory activity, and provides a new candidate compound for developing an anti-inflammatory drug.

The present disclosure provides a macrocyclic lactam compound as represented by formula 1 or a pharmaceutically acceptable salt thereof:

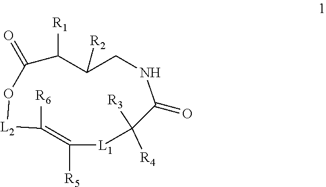

wherein R¹ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R² is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R³ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁴ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
L¹ is

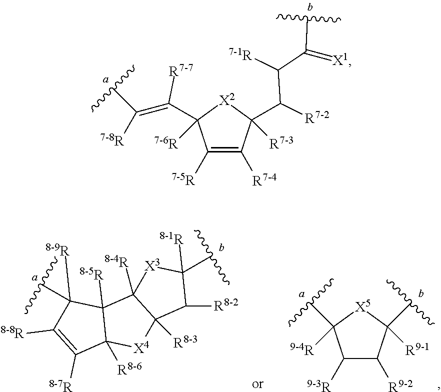

wherein the terminal a and R⁵ are connected to the same carbon atom;
X¹ is O, S or NH;
X² is O, S or NH;
R⁷⁻¹ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁷⁻² is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁷⁻³ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁷⁻⁴ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁷⁻⁵ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁷⁻⁶ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁷⁻⁷ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁷⁻⁸ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
X³ is O, S or NH;
X⁴ is O, S or NH;
R⁸⁻¹ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁸⁻² is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁸⁻³ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁸⁻⁴ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁸⁻⁵ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁸⁻⁶ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁸⁻⁷ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R¹⁻⁸ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁸⁻⁹ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
X⁵ is O, S or NH;
R⁹⁻¹ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁹⁻² is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁹⁻³ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁹⁻⁴ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁵ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁶ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
L² is

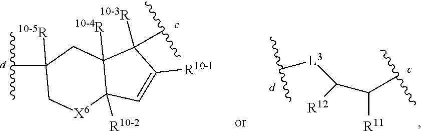

wherein the terminal c and R⁶ are connected to the same carbon atom;
X⁶ is O, S or NH;
R¹⁰⁻¹ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R¹⁰⁻² is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R¹⁰⁻³ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R¹⁰⁻⁴ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R¹⁰⁻⁵ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
L³ is a bond or methylene;
the above-mentioned 3- to 6-membered heterocycloalkyl independently contains 1, 2 or 3 heteroatoms; the above-mentioned 3- to 6-membered heterocycloalkyl contains 1, 2 or 3 heteroatoms independently selected from N, O and S; the above-mentioned 5- to 10-membered heteroaryl independently contains 1, 2 or 3 heteroatoms; the above-mentioned 5- to 10-membered heteroaryl contains 1, 2 or 3 heteroatoms independently selected from N, O and S.

In a certain embodiment, in the macrocyclic lactam compound as represented by formula 1 or a pharmaceutically acceptable salt thereof, some groups are defined as set forth below, and the remaining groups are defined as described in any additional embodiment (hereinafter referred to as “in a certain embodiment”),

wherein the macrocyclic lactam compound as represented by formula 1 is

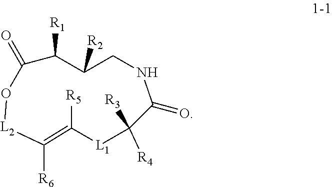

In a certain embodiment,

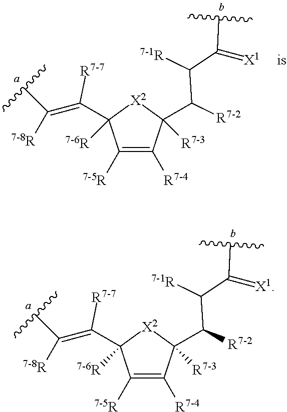

In a certain embodiment,

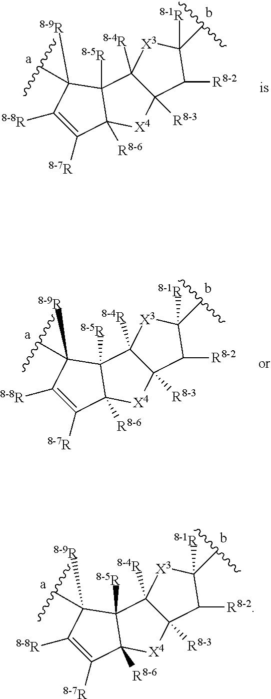

In a certain embodiment,

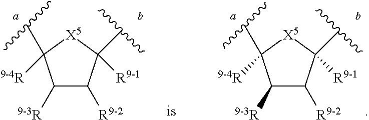

In a certain embodiment,

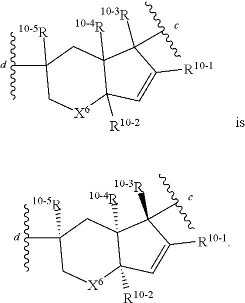

In a certain embodiment,

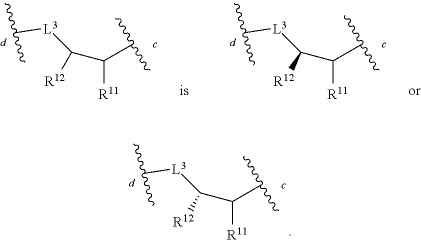

In a certain embodiment, the C₁-C₆ alkyl is methyl, ethyl, n-propyl, isopropyl, n-butyl, isobutyl, sec-butyl, tert-butyl or 2-methylbutyl.

In a certain embodiment, the C₂-C₆ alkenyl is ethenyl, 1-propenyl or 2-propenyl.

In a certain embodiment, the C₂-C₆ alkynyl is ethynyl, 1-propynyl or 2-propynyl.

In a certain embodiment, the C₁-C₆ alkoxy is methoxy, ethoxy, n-propoxy, isopropoxy, n-butoxy, isobutoxy, secbutoxy or tert-butoxy.

In a certain embodiment, the 3- to 6-membered cycloalkyl is cyclopropyl, cyclobutyl, cyclopentyl or cyclohexyl.

In a certain embodiment, the C₆-C₁₀ aryl is phenyl or naphthyl.

In a certain embodiment, the 3- to 6-membered heterocycloalkyl is tetrahydropyrrolyl, tetrahydrofuryl, morpholinyl, piperidyl or piperazinyl.

In a certain embodiment, the 5- to 10-membered heteroaryl is pyrrolyl, furyl, pyridyl, indolyl or quinolyl.

In a certain embodiment, the C₁-C₆ alkyl substituted by hydroxyl is hydroxymethyl.

In a certain embodiment, the macrocyclic lactam compound as represented by formula 1 is

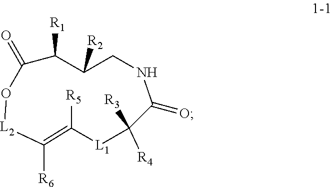

R¹ is C₁-C₆ alkoxy;
R² is hydroxyl;
R³ is hydroxyl;
R⁴ is C₁-C₆ alkyl;
L¹ is

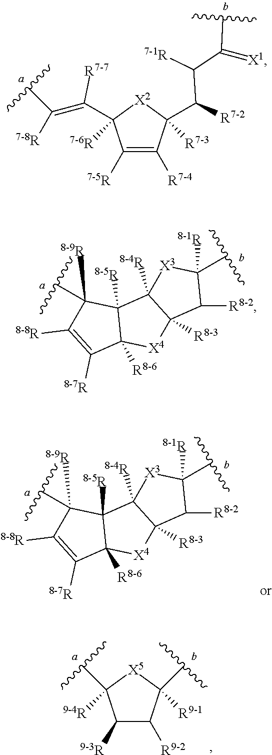

wherein the terminal a and R⁵ are connected to the same carbon atom;
X¹ is O;
R⁷⁻¹ is hydrogen;
R⁷⁻² is hydroxyl;
X² is O;
R⁷⁻³ is C₁-C₆ alkyl;
R⁷⁻⁴ is hydrogen;
R⁷⁻⁵ is C₁-C₆ alkyl;
R⁷⁻⁶ is hydrogen;
R⁷⁻⁷ is C₁-C₆ alkyl;
R⁷⁻⁸ is hydrogen;
X³ is O;
R⁸⁻¹ is hydroxyl;
R⁸⁻² is hydrogen;
R⁸⁻³ is hydrogen;
R⁸⁻⁴ is C₁-C₆ alkyl;
R⁸⁻⁵ is hydrogen;
R⁸⁻⁶ is C₁-C₆ alkyl;
R⁸⁻⁷ is hydrogen;
R⁸⁻⁸ is C₁-C₆ alkyl;
R⁸⁻⁹ is hydrogen;
X⁵ is O;
R⁹⁻¹ is hydroxyl;
R⁹⁻² is hydrogen;
R⁹⁻³ is hydroxyl;
R⁹⁻⁴ is C₁-C₆ alkyl;
R⁵ is hydrogen or C₁-C₆ alkyl;
R⁶ is hydrogen or C₁-C₆ alkyl;
L² is

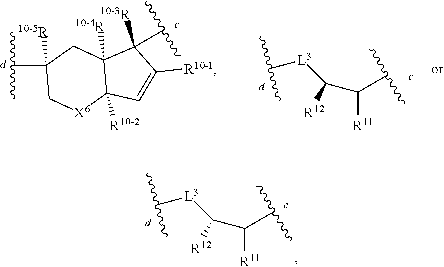

wherein the terminal c and R⁶ are connected to the same carbon atom;
X⁶ is O;
R¹⁰⁻¹ is C₁-C₆ alkyl;
R¹⁰⁻² is C₁-C₆ alkyl;
R¹⁰⁻³ is C₁-C₆ alkyl;
R¹⁰⁻⁴ is hydrogen;
R¹⁰⁻⁵ is hydrogen;
R¹¹ is hydrogen;
R¹² is hydroxyl or C₁-C₆ alkyl substituted by hydroxyl;
L³ is a bond or methylene.

In a certain embodiment, the macrocyclic lactam compound as represented by formula 1 is any one of the following structures:

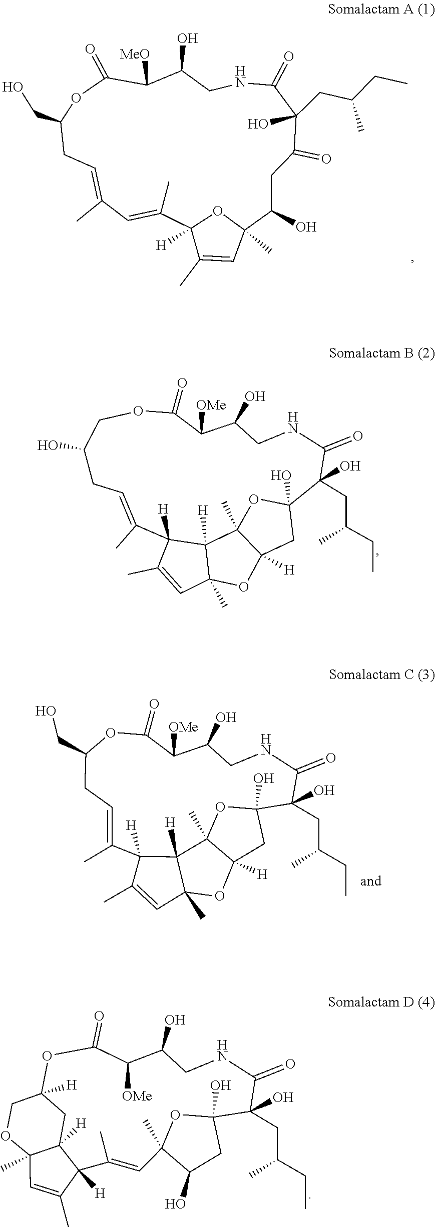

The present disclosure further provides a method for preparing the above-mentioned macrocyclic lactam compound as represented by formula 1, which comprises the following step: isolating the compound from the fermentation culture of Streptomyces somaliensis.

The strain Streptomyces somaliensis used in the preparation method can be purchased from Shanghai Boliang Sci & Tech Co., Ltd.

The fermentation culture in the preparation method is obtained using a medium having the following formula: 4 g of yeast extract, 10 g of malt extract, 4 g of glucose, 2.5 g of sea salt, 0.02% anti-forming agent, and 1 L of distilled water.

The fermentation culture in the preparation method is obtained using a medium having the following formula: 4 g of yeast extract (OXOID LP0021), 10 g of malt extract (OXOID LP0039), 4 g of glucose (HRBS-Q007), 2.5 g of sea salt (sea salt from Qingfengtang Co., Ltd.), 0.02% anti-forming agent (Hengxin Chemical Co., Ltd.), and 1 L of distilled water.

The fermentation temperature for the fermentation culture in the preparation method can be 28° C.

The fermentation conditions for the fermentation culture in the preparation method can be 28° C., 220 rpm.

The fermentation time for the fermentation culture in the preparation method can range from 3 to 7 days.

The isolation in the preparation method can sequentially comprise filtration, extraction, and column chromatography.

The filtration in the preparation method can be gauze filtration, as long as a fermentation broth is obtained.

The extraction in the preparation method can be performed with ethyl acetate.

The extraction in the preparation method can be performed three times with an equal volume of ethyl acetate.

The column chromatography in the preparation method can be performed once, twice, or three times.

In the preparation method, if the column chromatography is performed three times, the stationary-phase column for the first column chromatography can be a gel column.

In the preparation method, if the column chromatography is performed three times, the stationary-phase column for the first column chromatography can be a Sephadex LH-20 gel column.

In the preparation method, if the column chromatography is performed three times, the stationary-phase column for the first column chromatography can have the following size: diameter: 6 cm, and length: 150 cm.

In the preparation method, if the column chromatography is performed three times, the mobile phase for the first column chromatography can be dichloromethane and methanol.

In the preparation method, if the column chromatography is performed three times, the mobile phase for the first column chromatography can be CH₂Cl₂:MeOH=1:1.

In the preparation method, if the column chromatography is performed three times, the stationary-phase column for the second column chromatography can be a medium-pressure normal-phase silica gel column or medium-pressure reversed-phase silica gel column.

In the preparation method, if the column chromatography is performed three times, the stationary-phase column for the second column chromatography can be SEPAFLASH Silica Flash Column or SEPAFLASH SW 120 Bonded Spherical C18, 15 μm, 100 A [SW-5223-120-SP].

In the preparation method, if the column chromatography is performed three times, the mobile phase for the second column chromatography can be dichloromethane and methanol, or methanol and water.

In the preparation method, if the column chromatography is performed three times, the mobile phase for the second column chromatography can be dichloromethane-methanol (100:0, 30 min; 100:0-95:5, 210 min; 95:5-50:50, 30 min; 50:50-0:100, 30 min; and finally, flushing the column with pure methanol, or, 10%-100% for 5 h, then flushing the column with 100% methanol for 30 min).

In the preparation method, if the column chromatography is performed three times, the stationary-phase column for the third column chromatography can be a reversed-phase semi-preparative high performance liquid phase column.

In the preparation method, if the column chromatography is performed three times, the stationary-phase column for the third column chromatography can be YMC-Pack Pro C18 RS 250*10.0 mm L.D. S-5 μm, 8 nm, YMC-Pack Pro C18 RS 250*4.6 mm L.D. S-5 μm, 8 nm or Atlantis Prep T3 5 μm 10×250 mm column.

In the preparation method, if the column chromatography is performed three times, the mobile phase for the third column chromatography can be acetonitrile and water.

In the preparation method, if the column chromatography is performed three times, the mobile phase for the third column chromatography can be 45% acetonitrile/water (0.1% HCOOH), 42% acetonitrile/water, 55% acetonitrile/water or 90% acetonitrile/water.

The isolation in the preparation method can be any one of the following:
(1) filtering the fermentation culture with a gauze to obtain the fermentation broth, extracting the fermentation broth three times with an equal volume of ethyl acetate, and combining the extracts and concentrating same to obtain the ethyl acetate extract fraction;
subjecting the above-mentioned ethyl acetate extract fraction to chromatographic separation on a Sephadex LH-20 gel column (elution solvent: CH₂Cl₂:MeOH=1:1) to obtain components Fr. A-Fr. E;
subjecting the component Fr. B to chromatographic separation on a medium-pressure normal-phase silica gel column (gradient elution with dichloromethane-methanol) to obtain components Fr. B1-Fr. B8, and purifying the component Fr. B4 through a reversed-phase semi-preparative high performance liquid phase column (45% acetonitrile/water (0.1% HCOOH)) to obtain the compound somalactam A;
(2) filtering the fermentation culture with a gauze to obtain the fermentation broth, extracting the fermentation broth three times with an equal volume of ethyl acetate, and combining the extracts and concentrating same to obtain the ethyl acetate extract fraction;
subjecting the above-mentioned ethyl acetate extract fraction to chromatographic separation on a Sephadex LH-20 gel column (elution solvent: CH₂Cl₂:MeOH=1:1) to obtain components Fr. A-Fr. E;
subjecting the component Fr. B to chromatographic separation on a medium-pressure normal-phase silica gel column (gradient elution with dichloromethane-methanol) to obtain components Fr. B1-Fr. B8, and purifying the component Fr. B5 through a reversed-phase semi-preparative high performance liquid phase column (55% acetonitrile/water) to obtain the compound somalactam B;
(3) filtering the fermentation culture with a gauze to obtain the fermentation broth, extracting the fermentation broth three times with an equal volume of ethyl acetate, and combining the extracts and concentrating same to obtain the ethyl acetate extract fraction;
subjecting the above-mentioned ethyl acetate extract fraction to chromatographic separation on a Sephadex LH-20 gel column (elution solvent: CH₂Cl₂:MeOH=1:1) to obtain components Fr. A-Fr. E;
subjecting the component Fr. B to chromatographic separation on a medium-pressure normal-phase silica gel column (gradient elution with dichloromethane-methanol) to obtain components Fr. B1-Fr. B8, and purifying the component Fr. B6 through a reversed-phase semi-preparative high performance liquid phase column (90% acetonitrile/water) to obtain somalactams C or D, respectively;
(4) filtering the fermentation culture with a gauze to obtain the fermentation broth, extracting the fermentation broth three times with an equal volume of ethyl acetate, and combining the extracts and concentrating same to obtain the ethyl acetate extract fraction;
subjecting the above-mentioned ethyl acetate extract fraction to chromatographic separation on a Sephadex LH-20 gel column (elution solvent: CH₂Cl₂:MeOH=1:1) to obtain components Fr. A-Fr. E;
subjecting the component Fr. D to chromatographic separation on a medium-pressure reversed-phase silica gel column (gradient elution with methanol-water) to obtain components Fr. D1-Fr. D7, and purifying the component Fr. D3 through a reversed-phase semi-preparative high performance liquid phase column (42% acetonitrile/water) to obtain somalactams C or D, respectively.

The present disclosure further provides use of Streptomyces somaliensis in the preparation of the above-mentioned macrocyclic lactam compound as represented by formula 1.

The strain Streptomyces somaliensis used therein can be purchased from Shanghai Boliang Sci & Tech Co., Ltd.

The present disclosure further provides a pharmaceutical composition, comprising substance X and a pharmaceutical adjuvant, wherein the substance X is the above-mentioned macrocyclic lactam compound as represented by formula 1 or the pharmaceutically acceptable salt thereof.

The pharmaceutical composition can be a pharmaceutical composition for use in combating inflammation.

The pharmaceutical composition can be a pharmaceutical composition for use in inhibiting IL-6.

In the pharmaceutical composition, the amount of the substance X can be therapeutically effective.

The present disclosure further provides use of a substance X in the preparation of an anti-inflammatory drug, wherein the substance X is the above-mentioned macrocyclic lactam compound as represented by formula 1 or a pharmaceutically acceptable salt thereof.

The present disclosure further provides use of a substance X in the preparation of an IL-6 inhibitor, wherein the substance X is the above-mentioned macrocyclic lactam compound as represented by formula 1 or a pharmaceutically acceptable salt thereof.

The IL-6 inhibitor used therein can be an IL-6 inhibitor for use in vitro.

Unless otherwise specified, the terms used in the present disclosure have the following meanings:

The term “halogen” refers to fluorine, chlorine, bromine or iodine.

The term “alkyl” refers to linear or branched alkyl having a specified number of carbon atoms (e.g., C₁ to C₆). Alkyl includes, but is not limited to, methyl, ethyl, n-propyl, isopropyl, n-butyl, tert-butyl, isobutyl, sec-butyl, n-pentyl, n-hexyl, etc.

The term “alkoxy” refers to the group R^{X}—O—, wherein R^{X} is alkyl as defined above.

The term “cycloalkyl” refers to a saturated monocyclic cyclic group consisting only of carbon atoms and having a specified number of carbon atoms (e.g., C₃ to C₆). Cycloalkyl includes, but is not limited to, cyclopropyl, cyclobutyl, cyclopentyl, cyclohexyl, etc.

The term “heterocycloalkyl” refers to a cyclic group with a specified number of ring atoms (for example, 5- to 10-membered), a specified number of heteroatoms (for example, 1, 2 or 3) and specified heteroatom types (one or more of N, O and S), which is a saturated monocyclic ring. Heterocycloalkyl includes, but is not limited to, azetidinyl, tetrahydropyrrolyl, tetrahydrofuryl, morpholinyl, piperidyl, etc.

The term “aryl” refers to a cyclic group consisting only of carbon atoms and having a specified number of carbon atoms (for example, C₆ to C₁₀), which is a monocyclic ring or a polycyclic ring, where each ring contained therein is aromatic (according to Huckel's rule). Aryl includes, but is not limited to, phenyl, naphthyl, etc.

The term “heteroaryl” refers to a cyclic group with a specified number of ring atoms (for example, 5- to 10-membered), a specified number of heteroatoms (for example, 1, 2 or 3) and specified heteroatom types (one or more of N, O and S), which is a monocyclic ring or a polycyclic ring, where each ring contained therein is aromatic (according to Huckel's rule). Heteroaryl includes, but is not limited to, furyl, pyrrolyl, thienyl, pyrazolyl, imidazolyl, oxazolyl, thiazolyl, pyridyl, pyrimidinyl, indolyl, etc.

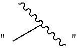

in a structural fragment means that the structural fragment is connected to other fragments in the molecule through this site. For example,

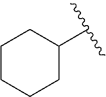 [SMILES: *C1CCCCC1]

refers to cyclohexyl.

The term “pharmaceutically acceptable salt” refers to a salt obtained by reacting a compound with a pharmaceutically acceptable (relatively non-toxic, safe, and suitable for use by patients) acid or base. When a compound contains relatively acidic functional groups, base addition salts can be obtained by contacting the free form of the compound with a sufficient amount of a pharmaceutically acceptable base in a suitable inert solvent. Pharmaceutically acceptable base addition salts include, but are not limited to, sodium salts, potassium salts, calcium salts, aluminum salts, magnesium salts, bismuth salts, ammonium salts, etc. When the compound contains relatively basic functional groups, acid addition salts can be obtained by contacting the free form of the compound with a sufficient amount of a pharmaceutically acceptable acid in a suitable inert solvent. Pharmaceutically acceptable acid addition salts include, but are not limited to, hydrochlorides, sulfates, methanesulfonates, etc. For details, see Handbook of Pharmaceutical Salts: Properties, Selection, and Use (P. Heinrich Stahl, 2002).

The term “therapeutically effective amount” refers to an amount of a compound that is sufficient to effectively treat a disease when administered to a patient. The therapeutically effective amount will vary depending on the compounds, disease type, disease severity, age of patients, etc., but can be adjusted by a person skilled in the art as appropriate.

The term “treat/treating/treatment” refers to any of the following: (1) alleviating one or more biological manifestations of a disease; (2) interfering with one or more points in the biological cascade leading to a disease; and (3) slowing down the progression of one or more biological manifestations of a disease.

The term “prevent/preventing/prevention” refers to reducing the risk of developing a disease.

The term “patient” refers to any animal, preferably a mammal, most preferably a human, who has been or is about to be treated. Mammals include, but are not limited to, cattle, horse, sheep, pig, cat, dog, mouse, rat, rabbit, guinea pig, monkey, human, etc.

The term “pharmaceutical adjuvant” refers to excipients and additives used in the production of drugs and formulation of prescriptions, and is all substances contained in a pharmaceutical preparation except for active ingredients. For details, see Pharmacopoeia of the People's Republic of China (2020 Edition) or Handbook of Pharmaceutical Excipients (Raymond C Rowe, 2009).

On the basis of not departing from common knowledge in the art, the above-mentioned various preferred conditions can be combined in any manner, such that various preferred examples of the present disclosure are obtained.

Reagents and raw materials used in the present disclosure are all commercially available.

The positive effects of the present disclosure lie in that the compound has a good inflammation inhibitory activity, and provides a new candidate compound for developing an anti-inflammatory drug.

### BRIEF DESCRIPTION OF THE DRAWINGS

FIG. 1 shows a single crystal diffraction pattern of Somalactam A.

FIG. 2 shows a single crystal diffraction pattern of Somalactam B.

FIG. 3 shows a single crystal diffraction pattern of Somalactam C.

FIG. 4 shows a single crystal diffraction pattern of Somalactam D.

### DETAILED DESCRIPTION OF THE PREFERRED EMBODIMENT

The present disclosure is further described below by way of examples; however, the present disclosure is not limited to the scope of the described examples. For the experimental methods in which no specific conditions are specified in the following examples, selections are made according to conventional methods and conditions or according to the product instructions.

In the following examples, Streptomyces somaliensis is purchased from Shanghai Boliang Sci & Tech Co., Ltd.

### Example 1 Preparation and Isolation of the Compound of the Present Disclosure

### 1. Preparation of Extracts

(1) Fermentation: Activated Streptomyces somaliensis was inoculated into a 250 mL triangular flask containing 100 mL of seed medium, and cultured on a shaker at 28° C., 220 rpm for 72 h to obtain a seed liquid, which was then inoculated into 48 1-L triangular flasks (inoculation amount: 10%, each containing 500 mL of fermentation medium, 24 L in total) and cultured with shaking on a shaker at 28° C., 220 rpm for 7 days to obtain the fermentation culture of the strain. The formula of the seed medium and fermentation medium was as follows: each liter of the media contained: 4 g of yeast extract (OXOID LP0021), 10 g of malt extract (OXOID LP0039), 4 g of glucose (HRBS-Q007), 2.5 g of sea salt (sea salt from Qingfengtang Co., Ltd.), 0.02% anti-forming agent (Hengxin Chemical Co., Ltd.), and 1 L of distilled water.

(2) Extraction: the fermentation broth was collected by filtering the fermentation culture with a gauze to remove the mycelium, and then extracted three times with an equal volume of ethyl acetate, and the extracts were combined and concentrated to obtain the ethyl acetate extract fraction.

### 2. Isolation and Purification

The above-mentioned ethyl acetate extract fraction was subjected to chromatographic separation on a Sephadex LH-20 gel column (diameter: 6 cm, length: 150 cm), and then eluted (solvent: CH₂Cl₂:MeOH=1:1) to obtain components Fr. A-Fr. E (the flow rate was controlled at 1 drop/s; a total of 40 tubes were used for the eluate (about 100 ml/30 min per tube); based on the analysis through thin layer chromatography (TLC), the eluates in tubes 1-9 were combined as Fr. A, tubes 10-26 as Fr. B, tubes 27-30 as Fr. C, tubes 31-35 as Fr. D, and tubes 36-40 as Fr. E).

Fr. B was subjected to chromatographic separation on a medium-pressure normal-phase silica gel column (SEPAFLASH Silica Flash column, 120 g), and gradient elution was carried out using dichloromethane-methanol (100:0, 30 min; 100:0-95:5, 210 min; 95:5-50:50, 30 min; 50:50-0:100, 30 min; and finally, the column was flushed with pure methanol; flow rate: 25 mL/min). The eluates were combined based on the analysis results from thin layer chromatography (TLC) to obtain components Fr. B1-Fr. B8 (collecting about 100 mL each, and then combining the corresponding eluates after analysis with TLC plate). Fr. B4 (retention time: 114 min-142 min, polar position: 2%-2.6% methanol/water), Fr. B5 (retention time: 142 min-170 min, polar position: 2.6%-3.3% methanol/water), and Fr. B6 (retention time: 170 min-198 min, polar position: 3.3%-4% methanol/water). Fr. B4 was purified (45% acetonitrile/water (0.1% HCOOH), flow rate: 1.0 mL/min, detection wavelength: 210 nm) through a reversed-phase semi-preparative high performance liquid phase column (YMC-Pack Pro C18 RS 250*4.6 mm L.D. S-5 μm, 8 nm) to obtain compound somalactam A (retention time: 11 min); Fr. B5 was purified (55% acetonitrile/water, flow rate: 3.0 mL/min, detection wavelength: 210 nm) through a reversed-phase semi-preparative high performance liquid phase column (Atlantis Prep T3 5 μm 10×250 mm column) to obtain compound somalactam B (retention time: 40 min); and Fr. B6 was purified (90% acetonitrile/water, flow rate: 3.0 mL/min, detection wavelength: 210 nm) through a reversed-phase semi-preparative high performance liquid phase column (Atlantis Prep T3 5 μm 10×250 mm Column) to respectively obtain compounds 3 and 4 (retention time: 6 min and 7 min), i.e., somalactams C and D.

Fr. D was subjected to chromatographic separation on a medium-pressure reversed-phase silica gel column to obtain components Fr. D1-Fr. D7 (SEPAFLASH SW 120 Bonded Spherical C18, 15 μm, 100 A [SW-5223-120-SP], mobile phase: methanol-water system, 10%-100% for 5 h, then flushing the column with 100% methanol for 30 min, flow rate: 25 ml/min, and combining based on the peaks); and Fr. D3 (retention time: 226 min, polar range: 78% methanol/water) was separated (42% acetonitrile/water, flow rate: 3.0 mL/min, detection wavelength: 210 nm) through a reversed-phase semi-preparative high performance liquid phase column (YMC-Pack Pro C18 RS 250*10.0 mm L.D. S-5 μm, 8 nm) to obtain somalactams C and D (retention time: 18 min and 22 min).

### 3. Structure Identification

The NMR, HRESIMS, IR, and UV determination data of compounds Somalactam A-D are as shown below:

Somalactam A (1): white amorphous solid; [α]_{D}²⁵ 0.53 (c 0.2, MeOH); UV (MeOH) λₘₐₓ (log ε) 195 (3.08) nm; HRESIMS m/z 580.3130 [M-H]⁻ (C₃₀H₄₆NO₁₀, calculated value: 580.3122) and m/z 604.3107 [M+Na]⁺ (C₃₀H₄₇NO₁₀Na⁺, theoretical value: 604.3098). IR (KBr) vmax: 3375, 2919, 1726, 1658, 1650, 1642, 1631, 1530, 1461, 1441, 1379, 1281, 1237, 1191, 1138, 1056, 982, 588, 538, 481, 461, 447, 415. ¹³C NMR data (DMSO-d₆) are as shown in the table below:

| Position | δ_{C}, type |
|---|---|
| 1 | 170.7, C |
| 2 | 83.7, C |
| 3 | 207.6, C |
| 4a | 39.7, CH₂ |
| 4b | |
| 5 | 70.3, CH |
| 6 | 89.9, C |
| 7 | 127.8, CH |
| 8 | 137.1, C |
| 9 | 92.2, CH |
| 10 | 132.8, C |
| 11 | 132.5, CH |
| 12 | 134.0, C |
| 13 | 124.9, CH |
| 14a | 28.8, CH₂ |
| 14b | |
| 15 | 75.4, CH |
| 16 | 169.7, C |
| 17 | 82.2, CH |
| 18 | 70.5, CH |
| 19a | 42.2, CH₂ |
| 19b | |
| 20a | 41.0, CH₂ |
| 20b | |
| 21 | 29.3, CH |
| 22a | 29.8, CH₂ |
| 22b | |
| 23 | 11.0, CH₃ |
| 24 | 20.5, CH₃ |
| 25 | 23.7, CH₃ |
| 26 | 12.3, CH₃ |
| 27 | 14.5, CH₃ |
| 28 | 17.3, CH₃ |
| 29 | 62.3, CH₂ |
| 30 | 57.7, OCH₃ |
| 2-OH | — |
| 5-OH | — |
| 18-OH | — |
| 29-OH | |
| NH | — |

Somalactam B (2): white amorphous solid; [α]_{D}²⁵ 53 (c 0.5, MeOH); UV (MeOH) λₘₐₓ (log ε) 193 (2.48) nm; HRESIMS m/z 580.3136 [M-H]⁻ (C₃₀H₄₆NO₁₀, calculated value: 580.3122). IR (KBr) vmax: 3380, 2963, 2880, 2271, 2145, 1748, 1624, 1537, 1414, 1376, 1333, 1262, 1250, 1197, 1110, 1088, 1070, 1021, 867, 804, 686, 611, 529, 477. ¹H and ¹³C NMR data (DMSO-d₆) are as shown in the table below:

| Position | δ_{C} | δ_{H}, mult. (J in Hz) |
|---|---|---|
| 1 | 179.0, C | — |
| 2 | 78.2, C | — |
| 3 | 110.3, C | — |
| 4a | 38.4, CH₂ | 2.36, d (15.1) |
| 4b | | 2.02, dd (15.0, 4.3) |
| 5 | 86.3, CH | 4.38, d (4.7) |
| 6 | 91.9, C | |
| 7 | 96.0, C | |
| 8 | 129.0 CH | 5.40, s |
| 9 | 146.8, C | |
| 10 | 58.4, CH | 3.65, s |
| 11 | 62.4, CH | 2.06, d (3.9) |
| 12 | 135.6, C | |
| 13 | 123.6, CH | 5.50, dd (11.1, 4.2) |
| 14a | 36.8, CH₂ | 2.56, t (12.6) |
| 14b | | 1.90, m |
| 15 | 68.0, CH | 3.49, m |
| 16a | 69.6, CH₂ | 4.14, dt (10.7, 2.2) |
| 16b | | 3.89, t (10.1) |
| 17 | 169.2 | |
| 18 | 81.3, CH | 3.76, br.s |
| 19 | 73.0, CH | 3.99, m |
| 20a | 42.6, CH₂ | 3.47, m |
| 20b | — | 3.14, m |
| 21a | 41.5, CH₂ | 1.89, dd (13.8, 5.4) |
| 21b | | 1.18, dd, (11.5, 5.0) |
| 22 | 29.9, CH | 1.39, m |
| 23a | 29.8, CH₂ | 1.15, m |
| 23b | | 0.99, m |
| 24 | 11.6, CH₃ | 0.74, t (7.4) |
| 25 | 21.8, CH₃ | 0.90, d (6.5) |
| 26 | 24.4, CH₃ | 1.26, s |
| 27 | 24.8, CH₃ | 1.30, s |
| 28 | 15.3, CH₃ | 1.56, s |
| 29 | 11.3, CH₃ | 1.33, s |
| 30 | 58.6, CH₃ | 3.34, s |
| 2-OH | — | 3.75, s |
| 3-OH | — | 6.24, s |
| 15-OH | — | 5.07, br.s |
| 19-OH | — | 4.73, d (6.0) |
| NH | — | 8.62, t (6.3) |

Somalactam C (3): white amorphous solid; [α]_{D}²⁵ 27.5 (c 0.5, MeOH); UV (MeOH) λₘₐₓ (log ε) 198 (2.46) nm; HRESIMS m/z 580.3127 [M-H]⁻ (C₃₀H₄₆NO₁₀, calculated value: 580.3122) and m/z 604.3099 [M+Na]⁺ (C₃₀H₄₇NO₁₀Na⁺, calcd. 604.3098). IR (KBr) vmax: 3381, 2962, 2930, 2874, 2262, 2131, 1732, 1643, 1526, 1438, 1373, 1331, 1282, 1243, 1194, 1130, 1108, 1058, 1026, 993, 964, 930, 911, 857, 834, 768, 735, 700, 654, 604, 517, 485. ¹H and ¹³C NMR data (DMSO-d₆) are as shown in the table below:

| Position | δ_{C} | δ_{H}, mult. (J in Hz) |
|---|---|---|
| 1 | 176.7, C | — |
| 2 | 78.3, C | — |
| 3 | 109.9, C | — |
| 4a | 39.1, CH₂ | 2.36, m |
| 4b | | 2.02, dd, |
| | | (12.0, 4.0) |
| 5 | 84.7, CH | 3.92, d, (4.0) |
| 6 | 94.9, C | |
| 7 | 96.0, C | |
| 8 | 130.6, CH | 5.22, s |
| 9 | 143.0, C | |
| 10 | 58.4, CH | 3.12, s |
| 11 | 64.9, CH | 1.89, br.s |
| 12 | 138.4, C | |
| 13 | 123.8, CH | 5.16, dd, |
| | | (9.5, 3.0) |
| 14a | 30.5, CH₂ | 2.39, m |
| 14b | | 2.22, d (12.0) |
| 15 | 73.8, CH | 5.02, m |
| 16 | 171.1, C | |
| 17 | 79.9, CH | 3.76, br s |
| 18 | 67.6, CH | 4.22, m |
| 19a | 41.2, CH₂ | 3.26, m |
| 19b | | 2.96, m |
| 20a | 40.3, CH₂ | 2.00, dd (11.5, 5.0) |
| 20b | | 1.18, dd (11.5, 5.0) |
| 21 | 30.0, CH | 1.46, m |
| 22a | 29.2, CH₂ | 1.25, m |
| 22b | | 1.02, m |
| 23 | 10.9, CH₃ | 0.76, t (6.5) |
| 24 | 20.6, CH₃ | 0.90, d (5.5) |
| 25 | 20.5, CH₃ | 1.27, s |
| 26 | 26.0, CH₃ | 1.50, s |
| 27 | 15.3, CH₃ | 1.56, s |
| 28 | 12.8, CH₃ | 1.40, s |
| 29 | 63.0, CH₂ | 3.49, m |
| 30 | 59.6, CH₃ | 3.46, s |
| 2-OH | — | 4.12, s |
| 29-OH | — | 4.88, br.s |
| 3-OH | — | 6.02, s |
| NH | — | 8.52, t, (5.5) |
| 18-OH | — | 4.62, d, (6.1) |

Somalactam D (4): white amorphous solid; [α]_{D}²⁵ 15.5 (c 0.5, MeOH); HRESIMS m/z 566.2974 [M-H]⁻ (C₂₉H₄₄NO₁₀, calculated value: 566.2965); UV (MeOH) λₘₐₓ (log ε) 200 (2.55) nm; IR (KBr) vmax: 3384, 2957, 2871, 1735, 1646, 1525, 1497, 1440, 1373, 1332, 1283, 1248, 1194, 1130, 1108, 1076, 1055, 1005, 957, 931, 912, 858, 837, 809, 731, 695, 654, 598, 517, 485, 466, 437. ¹H and ¹³C NMR data (DMSO-d₆) are as shown in the table below:

| Position | δ_{C} | δ_{H}, mult. (J in Hz) |
|---|---|---|
| 1 | 176.9, C | — |
| 2 | 78.9, C | — |
| 3 | 108.6, C | — |
| 4 | 42.6, CH₂ | 2.27, m |
| 5 | 75.2, CH | 4.21, t (3.7) |
| 6 | 87.7, C | |
| 7 | 134.2, CH | 5.34, s |
| 8 | 131.5 C | |
| 9 | 63.3, CH | 3.35 |
| 10 | 146.6, C | |
| 11 | 130.4, CH | 5.31, t (2.1) |
| 12 | 82.3, C | |
| 13 | 43.0, CH | 1.59, m |
| 14a | 23.0, CH₂ | 1.94, m |
| 14b | | 1.71, d (15.5) |
| 15 | 68.3, CH | 4.84, q (5.5) |
| 16 | 169.7, C | |
| 17 | 79.6, CH | 4.01, d (1.5) |
| 18 | 71.8, CH | 3.75, m |
| 19a | 40.6, CH | 3.63, m |
| 19b | | 2.99, m |
| 20a | 40.8, CH₂ | 2.01, dd (5.8) |
| 20b | — | 1.22, m |
| 21 | 29.8, CH | 1.44, m |
| 22a | 29.3, CH₂ | 1.24, m |
| 22b | | 1.03, m |
| 23 | 10.9, CH₃ | 0.75, t (7.2) |
| 24 | 20.8, CH₃ | 0.91, d (6.7) |
| 25 | 24.7, CH₃ | 1.33, s |
| 26 | 12.3, CH₃ | 1.39, s |
| 27 | 14.8, CH₃ | 1.63, s |
| 28 | 23.2, CH₃ | 1.26, s |
| 29a | 61.3, CH₂ | 3.82, dd |
| 29b | | 3.30, m |
| 30 | 57.8, CH₃ | 3.36, s |
| 2-OH | — | 5.29, s |
| 3-OH | — | 5.88, s |
| 5-OH | — | 5.75, d (4.0) |
| 18-OH | — | 4.70, d (6.3) |
| NH | — | 8.55, dd (3.2) |

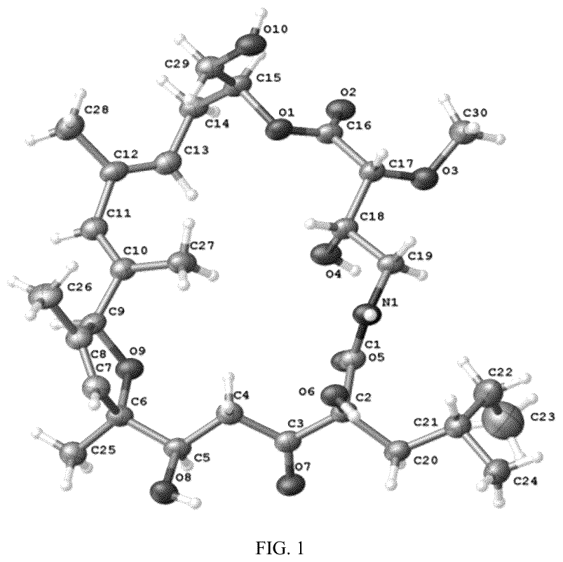

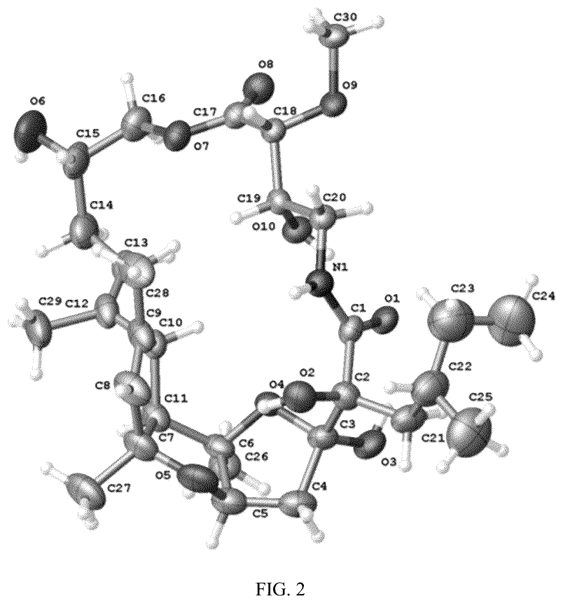

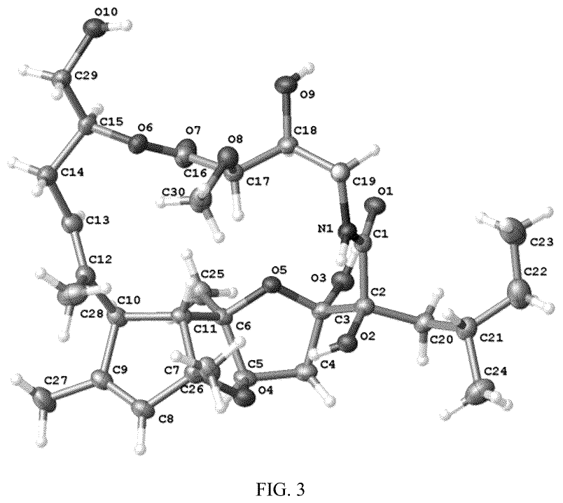

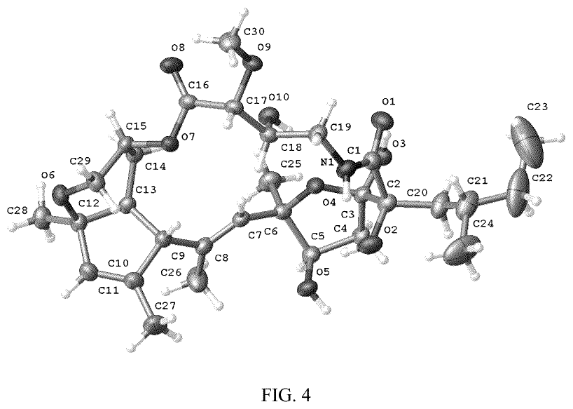

Furthermore, solvents (methanol/water 1:1) were used for crystallization of the above-mentioned compounds, i.e., somalactams A-D (Somalactam A: solvent system: methanol:water 2:1; the sample was first dissolved in 800 μl of methanol in a 10 mL vial and then 400 μl of purified water was added dropwise; after the mixture was uniformly mixed, the vial was placed in a cabinet (at room temperature); Somalactam B: solvent system: methanol:water 2:1; the sample was first dissolved in 800 μl of methanol in a 10 mL vial and then 400 μl of purified water was added dropwise; after the mixture was uniformly mixed, the vial was placed in a refrigerator at −4° C.; Somalactam C: solvent system: dichloromethane:methanol:water 5:5:1; the sample was first dissolved in 500 μl of dichloromethane in a 10 mL vial and then 500 μl of methanol and 100 μl of purified water were added dropwise; after the mixture was uniformly mixed, the vial was placed in a cabinet (at room temperature)). The respective single crystals were obtained, and single crystal diffraction was performed. The results were as shown in FIG. 1, FIG. 2, FIG. 3 and FIG. 4. With the aid of the nuclear magnetic resonance technique (¹H NMR, ¹³C NMR, DEPT, COSY, HSQC, HMBC, and ROESY), the absolute configurations of Somalactams A-D were determined.

To sum up, the chemical structures of Somalactams A-D are as follows:

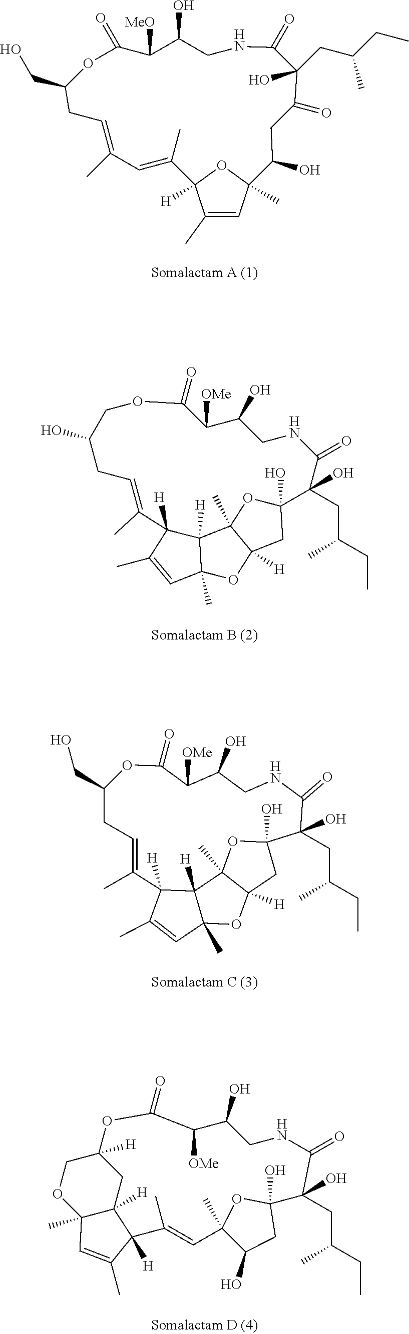

### Example 2 Anti-Inflammatory Activity Experiment of the Compound of the Present Disclosure

Culture of THP-1 cells and treatment of THP-1 cells with test compounds in combination with LPS

THP-1 cells (from the Cell Bank of the Chinese Academy of Sciences) were cultured in RPMI-1640 medium containing 10% FBS in a 5% CO₂ incubator at 37° C. 100 μL of a cell suspension in a complete culture medium containing antibiotics (100 μg/mL streptomycin and 100 U/mL penicillin) and PMA (final concentration: 160 nM) was added to each well in a 96-well plate (about 5×10⁴ cells/well). After 36 h of treatment, the cells achieved adherent growth.

The PMA-containing medium was discarded and then the cells were washed with 1×PBS solution. Serum-free RPMI-1640 (2 mL) was added and the cells were treated for 12 h. The active compounds to be tested were diluted into five concentration gradients and added to each well for a 2-hour treatment. The culture solutions containing the compounds were retained and the cells were treated with a serum-free medium (2 μL) containing LPS (1 mg/mL) for 24 h.

### Cytokine Content Assay

The content assay for cytokine IL-6 in the culture solution was performed according to the Human Inflammation Cytometric Bead Array (CBA) method in the manufacturer's instructions. After the above-mentioned cytokine levels were determined on the FACSCalibur flow cytometer, the FCAP Array software was used to analyze the results. The IC₅₀ value of the control drug was 6.02±0.12 μM.

Half maximal inhibitory concentration (IC₅₀) value (M) of compounds 1-4 on inflammatory cytokine IL-6

| Compound | IL-6 (μM) |
|---|---|
| 1 | 10.54 ± 0.06 |
| 2 | 11.12 ± 0.13 |
| 3 | 5.76 ± 1.18 |
| 4 | 9.12 ± 0.15 |
| Control drug Tocilizumab | 9.22 ± 0.12 |

It can be seen from the Table above that the four compounds all have inhibitory activities against the inflammatory cytokine IL-6, which are superior to or equivalent to that of the positive control drug Tocilizumab. The present disclosure provides anew lead compound for the development of new anti-inflammatory drugs, which holds promise as a new anti-inflammatory drug.

1. A macrocyclic lactam compound as represented by formula 1 or a pharmaceutically acceptable salt thereof: 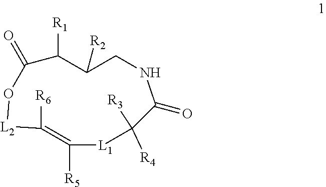
wherein R¹ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R² is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R³ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁴ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
L¹ is 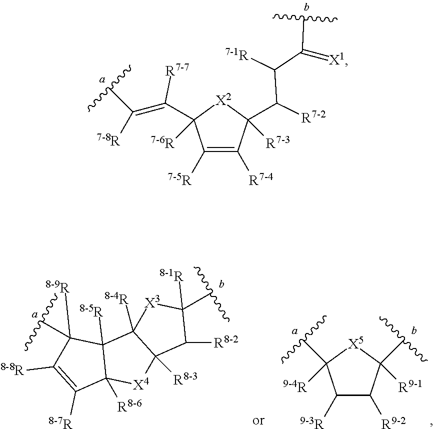
wherein the terminal a and R⁵ are connected to the same carbon atom;
X¹ is O, S or NH;
X² is O, S or NH;
R⁷⁻¹ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁷⁻² is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁷⁻³ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁷⁻⁴ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁷⁻⁵ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁷⁻⁶ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁷⁻⁷ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁷⁻⁸ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
X³ is O, S or NH;
X⁴ is O, S or NH;
R⁸⁻¹ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁸⁻² is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁸⁻³ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁸⁻⁴ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁸⁻⁵ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁸⁻⁶ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁸⁻⁷ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁸⁻⁸ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁸⁻⁹ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
X⁵ is O, S or NH;
R⁹⁻¹ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁹⁻² is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁹⁻³ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁹⁻⁴ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁵ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R⁶ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
L² is 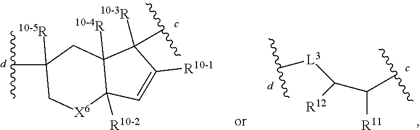
wherein the terminal c and R⁶ are connected to the same carbon atom;
X⁶ is O, S or NH;
R¹⁰⁻¹ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R¹⁰⁻² is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R¹⁰⁻³ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R¹⁰⁻⁴ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R¹⁰⁻⁵ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
L³ is a bond or methylene;
R¹¹ is hydrogen, hydroxyl, amino, cyano, halogen, C₁-C₆ alkyl, C₂-C₆ alkenyl, C₂-C₆ alkynyl, C₁-C₆ alkoxy, 3- to 6-membered cycloalkyl, C₆-C₁₀ aryl, 3- to 6-membered heterocycloalkyl or 5- to 10-membered heteroaryl;
R¹² is hydroxyl, C₁-C₆ alkoxy or C₁-C₆ alkyl substituted by hydroxyl;
the above-mentioned 3- to 6-membered heterocycloalkyl independently contains 1, 2 or 3 heteroatoms; the above-mentioned 3- to 6-membered heterocycloalkyl contains 1, 2 or 3 heteroatoms independently selected from N, O and S; the above-mentioned 5- to 10-membered heteroaryl independently contains 1, 2 or 3 heteroatoms; the above-mentioned 5- to 10-membered heteroaryl contains 1, 2 or 3 heteroatoms independently selected from N, O and S.

2. The macrocyclic lactam compound as represented by formula 1 or the pharmaceutically acceptable salt thereof of claim 1, wherein, the macrocyclic lactam compound as represented by formula 1 satisfies one or more of the following conditions: 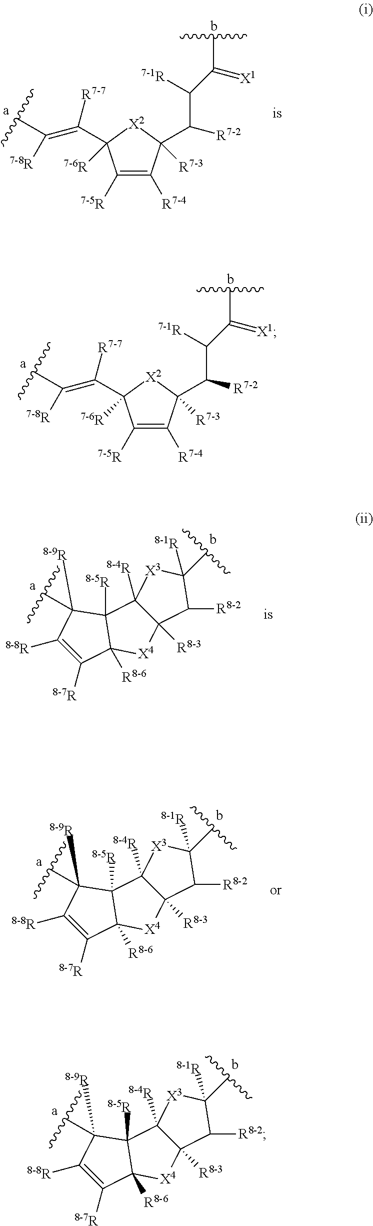 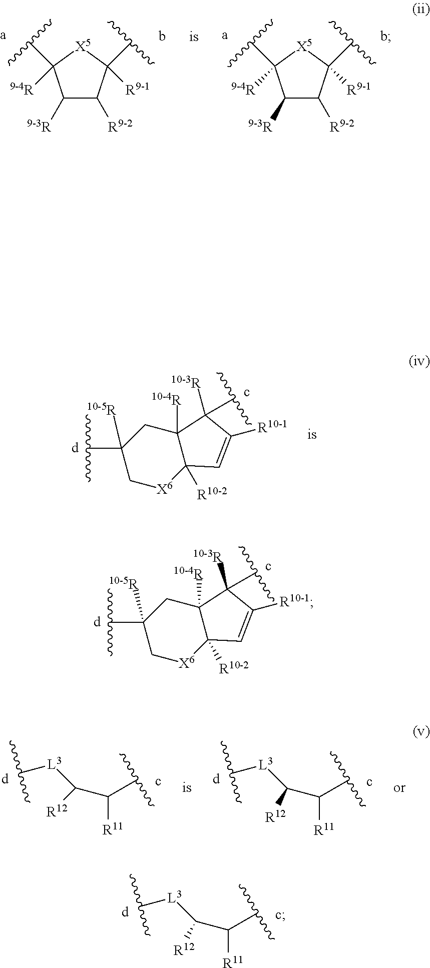
(vi) the C₁-C₆ alkyl is methyl, ethyl, n-propyl, isopropyl, n-butyl, isobutyl, sec-butyl, tert-butyl or 2-methylbutyl;
(vii) the C₂-C₆ alkenyl is ethenyl, 1-propenyl or 2-propenyl;
(viii) the C₂-C₆ alkynyl is ethynyl, 1-propynyl or 2-propynyl;
(ix) the C₁-C₆ alkoxy is methoxy, ethoxy, n-propoxy, isopropoxy, n-butoxy, isobutoxy, secbutoxy or tert-butoxy;
(x) the 3- to 6-membered cycloalkyl is cyclopropyl, cyclobutyl, cyclopentyl or cyclohexyl;
(xi) the C₆-C₁₀ aryl is phenyl or naphthyl;
(xii) the 3- to 6-membered heterocycloalkyl is tetrahydropyrrolyl, tetrahydrofuryl, morpholinyl, piperidyl or piperazinyl;
(xiii) the 5- to 10-membered heteroaryl is pyrrolyl, furyl, pyridyl, indolyl or quinolyl;
(ix) the C₁-C₆ alkyl substituted by hydroxyl is hydroxymethyl;
(xv) the macrocyclic lactam compound as represented by formula 1 is

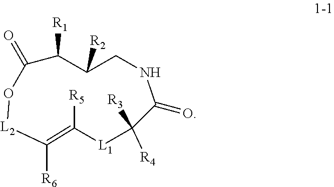

3. The macrocyclic lactam compound as represented by formula 1 or the pharmaceutically acceptable salt thereof of claim 1, wherein, the macrocyclic lactam compound as represented by formula 1 is: 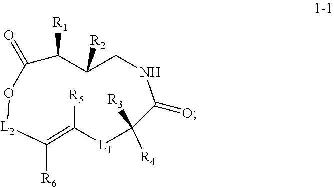
R¹ is C₁-C₆ alkoxy;
R² is hydroxyl;
R³ is hydroxyl;
R⁴ is C₁-C₆ alkyl;
L¹ is 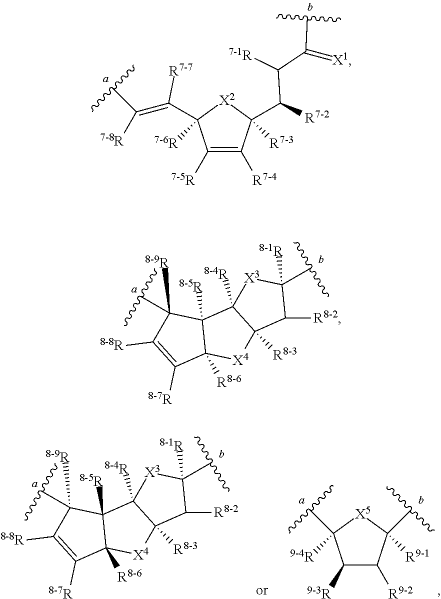
wherein the terminal a and R⁵ are connected to the same carbon atom;
X¹ is O;
R⁷⁻¹ is hydrogen;
R⁷⁻² is hydroxyl;
X² is O;
R⁷⁻³ is C₁-C₆ alkyl;
R⁷⁻⁴ is hydrogen;
R⁷⁻⁵ is C₁-C₆ alkyl;
R⁷⁻⁶ is hydrogen;
R⁷⁻⁷ is C₁-C₆ alkyl;
R⁷⁻⁸ is hydrogen;
X³ is O;
R⁸⁻¹ is hydroxyl;
R⁸⁻² is hydrogen;
R⁸⁻³ is hydrogen;
R⁸⁻⁴ is C₁-C₆ alkyl;
R⁸⁻⁵ is hydrogen;
R⁸⁻⁶ is C₁-C₆ alkyl;
R⁸⁻⁷ is hydrogen;
R⁸⁻⁸ is C₁-C₆ alkyl;
R⁸⁻⁹ is hydrogen;
X⁵ is O;
R⁹⁻¹ is hydroxyl;
R⁹⁻² is hydrogen;
R⁹⁻³ is hydroxyl;
R⁹⁻⁴ is C₁-C₆ alkyl;
R⁵ is hydrogen or C₁-C₆ alkyl;
R⁶ is hydrogen or C₁-C₆ alkyl;
L² is 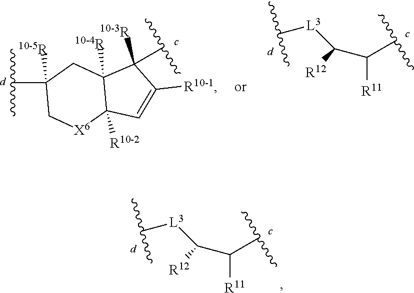
wherein the terminal c and R⁶ are connected to the same carbon atom;
X⁶ is O;
R¹⁰⁻¹ is C₁-C₆ alkyl;
R¹⁰⁻² is C₁-C₆ alkyl;
R¹⁰⁻³ is C₁-C₆ alkyl;
R¹⁰⁻⁴ is hydrogen;
R¹⁰⁻⁵ is hydrogen;
R¹¹ is hydrogen;
R¹² is hydroxyl or C₁-C₆ alkyl substituted by hydroxyl;
L³ is a bond or methylene.

4. The macrocyclic lactam compound as represented by formula 1 or the pharmaceutically acceptable salt thereof of claim 1, wherein, the macrocyclic lactam compound as represented by formula 1 is any one of the following structures:

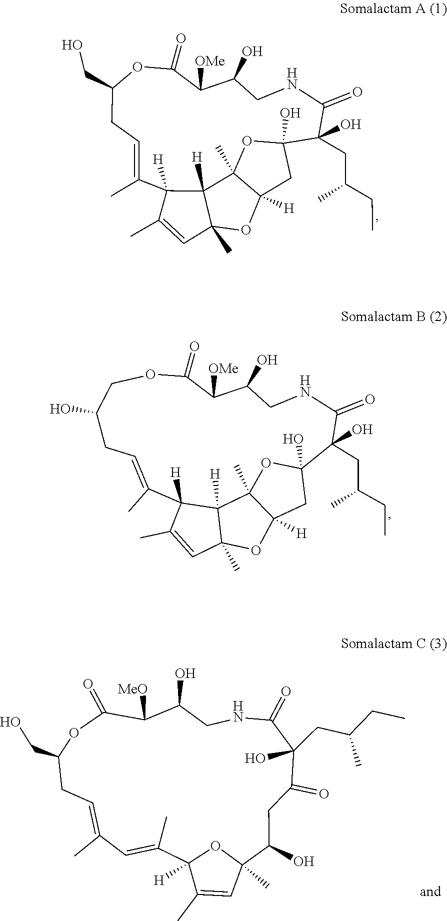 [SMILES: CC[C@H](C)C[C@@]1(O)C(=O)NC[C@H](O)[C@@H](OC)C(=O)O[C@H](CO)C/C=C(\C)[C@@H]2C(C)=C[C@@]3(C)O[C@@H]4C[C@@]1(O)O[C@]4(C)[C@@H]23 ; CC[C@H](C)C[C@@]1(O)C(=O)NC[C@H](O)[C@@H](OC)C(=O)OC[C@@H](O)C/C=C(\C)[C@H]2C(C)=C[C@]3(C)O[C@@H]4C[C@@]1(O)O[C@]4(C)[C@H]23 ; CC[C@H](C)C[C@]1(O)C(=O)C[C@@H](O)[C@@]2(C)C=C(C)[C@H](O2)/C(C)=C/C(C)=C/C[C@@H](CO)OC(=O)[C@H](OC)[C@@H](O)CNC1=O]

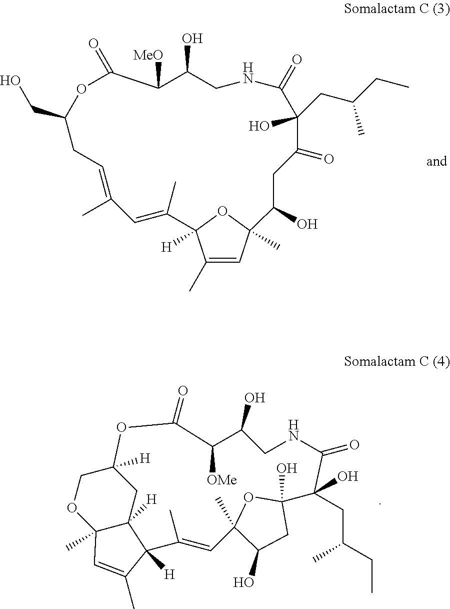

5. A method for preparing the macrocyclic lactam compound as represented by formula 1 as defined in claim 1, comprising the following step: isolating the compound from the fermentation culture of Streptomyces somaliensis.

6. The method for preparing the macrocyclic lactam compound as represented by formula 1 of claim 5, wherein, the method satisfies one or more of the following conditions:
(i) the strain Streptomyces somaliensis used in the method is purchased from Shanghai Boliang Sci & Tech Co., Ltd;
(ii) the fermentation culture is obtained using a medium having the following formula: 4 g of yeast extract, 10 g of malt extract, 4 g of glucose, 2.5 g of sea salt, 0.02% anti-forming agent, and 1 L of distilled water;
(iii) the fermentation temperature for the fermentation culture in the preparation method is 28° C.;
(iv) the fermentation conditions for the fermentation culture in the preparation method is 28° C., 220 rpm;
(v) the fermentation time for the fermentation culture in the preparation method ranges from 3 to 7 days;
(vi) the isolation in the preparation method sequentially comprises filtration, extraction, and column chromatography;
(vii) the filtration in the preparation method is gauze filtration, as long as a fermentation broth is obtained;
(viii) the extraction in the preparation method is performed with ethyl acetate;
(ix) the extraction in the preparation method is performed three times with an equal volume of ethyl acetate;
(x) the column chromatography in the preparation method is performed once, twice, or three times.

7. (canceled)

8. A pharmaceutical composition, comprising the macrocyclic lactam compound as represented by formula 1 or the pharmaceutically acceptable salt thereof as defined in claim 1 and a pharmaceutical adjuvant.

9-10. (canceled)

11. A pharmaceutical composition, comprising the macrocyclic lactam compound defined in claim 4 and a pharmaceutical adjuvant.

12. An IL-6 inhibitor, comprising the macrocyclic lactam compound as represented by formula 1 or the pharmaceutically acceptable salt thereof as defined in claim 1.

13. The IL-6 inhibitor of claim 12, wherein the IL-6 inhibitor is used therein is an IL-6 inhibitor for use in vitro.

14. An IL-6 inhibitor, comprising the macrocyclic lactam compound or the pharmaceutically acceptable salt thereof as defined in claim 4.

15. The IL-6 inhibitor of claim 14, wherein the IL-6 inhibitor is used therein is an IL-6 inhibitor for use in vitro.

16. A method for treating an anti-inflammatory, comprising the macrocyclic lactam compound as represented by formula 1 or the pharmaceutically acceptable salt thereof as defined in claim 1.

17. A method for treating an anti-inflammatory, comprising the macrocyclic lactam compound or the pharmaceutically acceptable salt thereof as defined in claim 4.

18. The method of claim 6, wherein, the method satisfies one or more of the following conditions:
(i) in the preparation method, if the column chromatography is performed three times, the stationary-phase column for the first column chromatography is a gel column;
(ii) in the preparation method, if the column chromatography is performed three times, the stationary-phase column for the first column chromatography is a Sephadex LH-20 gel column;
(iii) in the preparation method, if the column chromatography is performed three times, the stationary-phase column for the first column chromatography has the following size: diameter: 6 cm, and length: 150 cm;
(iv) in the preparation method, if the column chromatography is performed three times, the mobile phase for the first column chromatography is dichloromethane and methanol;
(v) in the preparation method, if the column chromatography is performed three times, the mobile phase for the first column chromatography is CH₂Cl₂:MeOH=1:1;
(vi) in the preparation method, if the column chromatography is performed three times, the stationary-phase column for the second column chromatography is a medium-pressure normal-phase silica gel column or medium-pressure reversed-phase silica gel column;
(vii) in the preparation method, if the column chromatography is performed three times, the stationary-phase column for the second column chromatography is SEPAFLASH Silica Flash Column or SEPAFLASH SW 120 Bonded Spherical C18, 15 μm, 100 A [SW-5223-120-SP];
(viii) in the preparation method, if the column chromatography is performed three times, the mobile phase for the second column chromatography is dichloromethane and methanol, or methanol and water;
(ix) in the preparation method, if the column chromatography is performed three times, the mobile phase for the second column chromatography is dichloromethane-methanol (100:0, 30 min; 100:0-95:5, 210 min; 95:5-50:50, 30 min; 50:50-0:100, 30 min; and finally, flushing the column with pure methanol), or, 10%-100% for 5 h, then flushing the column with 100% methanol for 30 min);
(x) in the preparation method, if the column chromatography is performed three times, the stationary-phase column for the third column chromatography is a reversed-phase semi-preparative high performance liquid phase column;
in the preparation method, if the column chromatography is performed three times, the stationary-phase column for the third column chromatography can be YMC-Pack Pro C18 RS 250*10.0 mm L.D. S-5 μm, 8 nm, YMC-Pack Pro C18 RS 250*4.6 mm L.D. S-5 μm, 8 nm or Atlantis Prep T3 5 μm 10×250 mm column;
(xi) in the preparation method, if the column chromatography is performed three times, the mobile phase for the third column chromatography is acetonitrile and water;
(xii) in the preparation method, if the column chromatography is performed three times, the mobile phase for the third column chromatography is 45% acetonitrile/water (0.1% HCOOH), 42% acetonitrile/water, 55% acetonitrile/water or 90% acetonitrile/water.

19. A method for preparing the macrocyclic lactam compound of claim 4, comprising the following step: isolating the compound from the fermentation culture of Streptomyces somaliensis.

20. The method of claim 19, wherein, the method satisfies one or more of the following conditions:
(i) the strain Streptomyces somaliensis used in the method is purchased from Shanghai Boliang Sci & Tech Co., Ltd;
(ii) the fermentation culture is obtained using a medium having the following formula: 4 g of yeast extract, 10 g of malt extract, 4 g of glucose, 2.5 g of sea salt, 0.02% anti-forming agent, and 1 L of distilled water;
(iii) the fermentation temperature for the fermentation culture in the preparation method is 28° C.;
(iv) the fermentation conditions for the fermentation culture in the preparation method is 28° C., 220 rpm;
(v) the fermentation time for the fermentation culture in the preparation method ranges from 3 to 7 days;
(vi) the isolation in the preparation method sequentially comprises filtration, extraction, and column chromatography;
(vii) the filtration in the preparation method is gauze filtration, as long as a fermentation broth is obtained;
(viii) the extraction in the preparation method is performed with ethyl acetate;
(ix) the extraction in the preparation method is performed three times with an equal volume of ethyl acetate;
(x) the column chromatography in the preparation method is performed once, twice, or three times.

21. The method of claim 20, wherein, the method satisfies one or more of the following conditions:
(i) in the preparation method, if the column chromatography is performed three times, the stationary-phase column for the first column chromatography is a gel column;
(ii) in the preparation method, if the column chromatography is performed three times, the stationary-phase column for the first column chromatography is a Sephadex LH-20 gel column;
(iii) in the preparation method, if the column chromatography is performed three times, the stationary-phase column for the first column chromatography has the following size: diameter: 6 cm, and length: 150 cm;
(iv) in the preparation method, if the column chromatography is performed three times, the mobile phase for the first column chromatography is dichloromethane and methanol;
(v) in the preparation method, if the column chromatography is performed three times, the mobile phase for the first column chromatography is CH₂Cl₂:MeOH=1:1;
(vi) in the preparation method, if the column chromatography is performed three times, the stationary-phase column for the second column chromatography is a medium-pressure normal-phase silica gel column or medium-pressure reversed-phase silica gel column;
(vii) in the preparation method, if the column chromatography is performed three times, the stationary-phase column for the second column chromatography is SEPAFLASH Silica Flash Column or SEPAFLASH SW 120 Bonded Spherical C18, 15 μm, 100 A [SW-5223-120-SP];
(viii) in the preparation method, if the column chromatography is performed three times, the mobile phase for the second column chromatography is dichloromethane and methanol, or methanol and water;
(ix) in the preparation method, if the column chromatography is performed three times, the mobile phase for the second column chromatography is dichloromethane-methanol (100:0, 30 min; 100:0-95:5, 210 min; 95:5-50:50, 30 min; 50:50-0:100, 30 min; and finally, flushing the column with pure methanol), or, 10%-100% for 5 h, then flushing the column with 100% methanol for 30 min);
(x) in the preparation method, if the column chromatography is performed three times, the stationary-phase column for the third column chromatography is a reversed-phase semi-preparative high performance liquid phase column;
in the preparation method, if the column chromatography is performed three times, the stationary-phase column for the third column chromatography can be YMC-Pack Pro C18 RS 250*10.0 mm L.D. S-5 μm, 8 nm, YMC-Pack Pro C18 RS 250*4.6 mm L.D. S-5 μm, 8 nm or Atlantis Prep T3 5 μm 10×250 mm column;
(xi) in the preparation method, if the column chromatography is performed three times, the mobile phase for the third column chromatography is acetonitrile and water;
(xii) in the preparation method, if the column chromatography is performed three times, the mobile phase for the third column chromatography is 45% acetonitrile/water (0.1% HCOOH), 42% acetonitrile/water, 55% acetonitrile/water or 90% acetonitrile/water.

22. The method of claim 19, wherein, the isolation is any one of the following:
(1) filtering the fermentation culture with a gauze to obtain the fermentation broth, extracting the fermentation broth three times with an equal volume of ethyl acetate, and combining the extracts and concentrating same to obtain the ethyl acetate extract fraction;
subjecting the above-mentioned ethyl acetate extract fraction to chromatographic separation on a Sephadex LH-20 gel column with an elution solvent: CH₂Cl₂:MeOH=1:1 to obtain components Fr. A-Fr. E;
subjecting the component Fr. B to chromatographic separation on a medium-pressure normal-phase silica gel column (gradient elution with dichloromethane-methanol) to obtain components Fr. B1-Fr. B8, and purifying the component Fr. B4 through a reversed-phase semi-preparative high performance liquid phase column with a mobile phase of 45% acetonitrile/water containing 0.1% HCOOH to obtain the compound somalactam C;
(2) filtering the fermentation culture with a gauze to obtain the fermentation broth, extracting the fermentation broth three times with an equal volume of ethyl acetate, and combining the extracts and concentrating same to obtain the ethyl acetate extract fraction;
subjecting the above-mentioned ethyl acetate extract fraction to chromatographic separation on a Sephadex LH-20 gel column with an elution solvent: CH₂Cl₂:MeOH=1:1 to obtain components Fr. A-Fr. E;
subjecting the component Fr. B to chromatographic separation on a medium-pressure normal-phase silica gel column (gradient elution with dichloromethane-methanol) to obtain components Fr. B1-Fr. B8, and purifying the component Fr. B5 through a reversed-phase semi-preparative high performance liquid phase column with a mobile phase of 55% acetonitrile/water to obtain the compound somalactam B;
(3) filtering the fermentation culture with a gauze to obtain the fermentation broth, extracting the fermentation broth three times with an equal volume of ethyl acetate, and combining the extracts and concentrating same to obtain the ethyl acetate extract fraction;
subjecting the above-mentioned ethyl acetate extract fraction to chromatographic separation on a Sephadex LH-20 gel column with an elution solvent: CH₂Cl₂:MeOH=1:1 to obtain components Fr. A-Fr. E;
subjecting the component Fr. B to chromatographic separation on a medium-pressure normal-phase silica gel column, gradient elution with dichloromethane-methanol, to obtain components Fr. B1-Fr. B8, and purifying the component Fr. B6 through a reversed-phase semi-preparative high performance liquid phase column with a mobile phase of 90% acetonitrile/water to obtain somalactams A and D, respectively;
(4) filtering the fermentation culture with a gauze to obtain the fermentation broth, extracting the fermentation broth three times with an equal volume of ethyl acetate, and combining the extracts and concentrating same to obtain the ethyl acetate extract fraction;
subjecting the above-mentioned ethyl acetate extract fraction to chromatographic separation on a Sephadex LH-20 gel column with an elution solvent: CH₂Cl₂:MeOH=1:1 to obtain components Fr. A-Fr. E;
subjecting the component Fr. D to chromatographic separation on a medium-pressure reversed-phase silica gel column, gradient elution with methanol-water, to obtain components Fr. D1-Fr. D7, and purifying the component Fr. D3 through a reversed-phase semi-preparative high performance liquid phase column with a mobile phase of 42% acetonitrile/water to obtain somalactams A and D, respectively.
